(19) United States
(12) Patent Application Publication
(10) Pub. No.: US 2021/0387180 A1
(43) Pub. Date: Dec. 16, 2021

(54) BLOOD EXAMINATION KIT AND METHOD OF SEPARATING PLASMA OR SERUM

(71) Applicant: FUJIFILM Corporation, Tokyo (JP)

(72) Inventors: Hirotoshi ONO, Ashigarakami-gun (JP); Makoto ISOZAKI, Ashigarakami-gun (JP); Keiichiro TAKAHASHI, Ashigarakami-gun (JP); Takuma KAWAMOTO, Ashigarakami-gun (JP); Osamu NOGUCHI, Ashigarakami-gun (JP)

(73) Assignee: FUJIFILM Corporation, Tokyo (JP)

(21) Appl. No.: 17/412,955

(22) Filed: Aug. 26, 2021

Related U.S. Application Data
(63) Continuation of application No. PCT/JP2020/013332, filed on Mar. 25, 2020.

(30) Foreign Application Priority Data
Mar. 27, 2019 (JP) 2019-060819
Aug. 26, 2019 (JP) 2019-153783

(51) Int. Cl. B01L 3/00; A61B 5/151; G01N 33/49

(52) U.S. Cl. CPC B01L 3/5023; A61B 5/151; G01N 33/49; B01L 2300/0609; B01L 2300/069; B01L 2300/04; B01L 2400/0622; B01L 2300/0681

(57) ABSTRACT

There are provided a blood examination kit with which the procedure carried out by an examination subject is simplified and a method of separating plasma or serum. The blood examination kit includes a blood collection instrument including a diluent and a first container that has an opening formed on one end side and accommodates the diluent; an absorption instrument including a first lid portion member that is arranged on one end side and can seal the opening of the first container, and an absorption member that is arranged on the other end side and absorbs a blood sample; and a separation and sealing instrument including a filtration member that separates plasma or serum from the blood sample diluted with the diluent, a second container that holds the filtration member on the other end side, accommodates the separated plasma or serum, and can be inserted into the first container, a second lid portion member that is arranged on one end side of the second container and can seal the opening of the first container, and a valve member that closes a flow channel between the first container and the second container.

### CROSS-REFERENCE TO RELATED APPLICATIONS

The present application is a Continuation of PCT International Application No. PCT/JP2020/013332 filed on Mar. 25, 2020 claiming priorities under 35 U.S.C § 119(a) to Japanese Patent Application No. 2019-060819 filed on Mar. 27, 2019 and Japanese Patent Application No. 2019-153783 filed on Aug. 26, 2019. Each of the above applications is hereby expressly incorporated by reference, in its entirety, into the present application.

### BACKGROUND OF THE INVENTION

### 1. Field of the Invention

The present invention relates to a blood examination kit and a method of separating plasma or serum.

### 2. Description of the Related Art

As a method of collecting a biological specimen for collecting and examining a biological specimen, for example, in the case of blood, there are a general blood collection in which a certain qualified person such as a doctor collects blood from a vein using a syringe and a self-blood collection in which an examination subject inserts a blood collection needle into a finger of the hand of the examination subject to collect blood.

The blood collected by the general blood collection is transported to a medical institution or an examination institution in a state of being sealed in a collection container, and the examination is carried out there. In a case where the blood sample is transported without being separated into plasma or serum, the examination is carried out after the blood sample is separated into blood cells and plasma by a centrifuge at the medical institution or the examination institution.

On the other hand, in the self-blood collection carried out by an examination subject, the examination subject separates the blood sample after blood collection into plasma or serum. The blood sample separated into plasma or serum is transported to an examination site and the blood sample is examined.

In a case where self-blood collection is carried out, the separation of the blood sample is carried out using a separation instrument. For example, JP2003-270239A discloses a biological specimen separation instrument that includes a biological specimen collection unit that accommodates a collected biological specimen, a filtering unit for causing a predetermined component in the collected biological specimen to pass, and a separated component accommodation unit that accommodates the predetermined component that has passed through the filtering unit.

### SUMMARY OF THE INVENTION

A blood examination kit including a separation instrument is required to reduce the complexity of manual work of an examination subject from the collection of a blood sample to the separation of plasma or serum.

The present invention has been made in consideration of such circumstances and provides a blood examination kit with which the complexity of manual work from the collection of a blood sample to the separation of plasma or serum can be reduced and a method of separating plasma or serum.

A blood examination kit according to a first aspect of the present invention includes a blood collection instrument including a diluent and a first container that has an opening formed on one end side and accommodates the diluent; an absorption instrument including a first lid portion member that is arranged on one end side and can seal the opening of the first container, and an absorption member that is arranged on the other end side and absorbs a blood sample; and a separation and sealing instrument including a filtration member that separates plasma or serum from the blood sample diluted with the diluent, a second container that holds the filtration member on the other end side, accommodates the separated plasma or serum, and can be inserted into the first container, a second lid portion member that is arranged on one end side of the second container and can seal the opening of the first container, and a valve member that closes a flow channel between the first container and the second container. According to the blood examination kit of the first aspect, the work carried out by an examination subject can be simplified.

In the second aspect of the present invention, the absorption instrument includes a holding member that holds the absorption member and a protruding shaft that is arranged on the one end side from the absorption member, and in a case where a first lid portion member of the absorption instrument seals the opening of the first container of the blood collection instrument, the protruding shaft pushes out the absorption member from the holding member into the diluent. In the second aspect, the absorption member can be reliably brought into contact with and the diluent, and the blood sample of the absorption member can be eluted into the diluent.

In the third aspect of the present invention, the absorption member of the absorption instrument is a fiber rod. In the third aspect, the absorption of the blood sample is facilitated.

In the fourth aspect of the present invention, the separation and sealing instrument includes a rod member that holds the valve member on the other end side, and after the second container of the separation and sealing instrument reaches the first container of the blood collection instrument, the second lid portion member of the separation and sealing instrument is rotated to move the rod member to a side of the filtration member, and the valve member that is held by the rod member closes the flow channel between the first container and the second container. In the fourth aspect, the diluent after filtration is prevented from moving from the second container to the first container.

In the fifth aspect of the present invention, the filtration member of the separation and sealing instrument is hydrophilic. In the fifth aspect of the present invention, the filtration of the blood sample is reliably carried out.

In the sixth aspect of the present invention, the filtration member of the separation and sealing instrument includes a depth filter and a membrane filter. In the sixth aspect, a preferred aspect of the filtration member is defined.

In the seventh aspect of the present invention, a lancet having a puncture needle is further provided. In the seventh aspect, the collection of the blood sample is facilitated.

In the eighth aspect of the present invention, a power transmission member that transmits a force from the second lid portion member to the second container is provided, and a protruding portion that is provided on the second container engages with a protruding portion that is provided on the power transmission member. In the eighth aspect of the present invention, a preferred aspect of the separation and sealing instrument is defined.

In the ninth aspect of the present invention, the power transmission member elastically deforms in a case where a torque of 0.1 Nm or more is applied. In the ninth aspect of the present invention, the preferred characteristics of the power transmission member is defined.

In the tenth aspect of the present invention, a compressive elastic member is provided between the second container and the second lid portion member. In the tenth aspect of the present invention, another preferred aspect of the separation and sealing instrument is defined.

In the eleventh aspect of the present invention, the compressive elastic member has a spring constant of 4,900 N/m or more. In the eleventh aspect of the present invention, the preferred characteristics of the compressive elastic member are defined.

A method of separating plasma or serum according to the twelfth aspect of the present invention includes a step of preparing a blood collection instrument including a diluent and a first container that has an opening formed on one end side and accommodates the diluent; a step of preparing an absorption instrument including a first lid portion member that is arranged on one end side and can seal the opening of the first container, and an absorption member that is arranged on the other end side and absorbs a blood sample and absorbing a blood sample into the absorption member; a step of mounting the absorption instrument on the blood collection instrument, sealing the opening of the first container of the blood collection instrument with the first lid portion member of the absorption instrument, and eluting the blood sample from the absorption member into the diluent; a step of removing the absorption instrument from the blood collection instrument; and a step of preparing a separation and sealing instrument including a filtration member, a second container that holds the filtration member on the other end side and can be inserted into the first container, a second lid portion member that is arranged on one end side of the second container and can seal the opening of the first container, and a valve member that closes a flow channel between the first container and the second container, inserting the second container of the separation and sealing instrument into the first container of the blood collection instrument to separate plasma or serum from the blood sample diluted with the diluent with the filtration member, accommodating the separated plasma or serum in the second container, and sealing the opening of the first container of the blood collection instrument with second lid portion member of the separation and sealing instrument to close the flow channel between the first container and the second container with the valve member. In the twelfth aspect, the work carried out by an examination subject can be simplified.

In the thirteenth aspect of the present invention, the step of eluting the blood sample includes pushing out the absorption member from the absorption instrument into the diluent. In the thirteenth aspect, the absorption member can be reliably brought into contact with and the diluent, and the blood sample of the absorption member can be eluted into the diluent.

According to the present invention, the work carried out by an examination subject can be simplified.

### BRIEF DESCRIPTION OF THE DRAWINGS

FIG. 1 is a configuration view illustrating an example of a blood examination kit.

FIG. 2 is a cross-sectional view of a blood collection instrument.

FIG. 3 is a cross-sectional view of an absorption instrument.

FIG. 4 is a cross-sectional view of a separation and sealing instrument.

FIG. 5 is a cross-sectional view of a lancet.

FIG. 6 is a view illustrating a procedure of a method of separating plasma or serum.

FIG. 7 is a view illustrating the procedure of the method of separating plasma or serum.

FIG. 8 is a view illustrating the procedure of the method of separating plasma or serum.

FIG. 9 is a view illustrating the procedure of the method of separating plasma or serum.

FIG. 10 is a view illustrating the procedure of the method of separating plasma or serum.

FIG. 11 is a view illustrating the procedure of the method of separating plasma or serum.

FIG. 12 is a view illustrating the procedure of the method of separating plasma or serum.

FIG. 13 is a view illustrating the procedure of the method of separating plasma or serum.

FIG. 14 is a view illustrating a procedure of the method of separating plasma or serum.

FIG. 15 is a view illustrating the procedure of the method of separating plasma or serum.

FIG. 16 is a view illustrating the procedure of the method of separating plasma or serum.

FIG. 17 is an assembly perspective view of the blood collection instrument and the separation and sealing instrument.

FIG. 18 is a view illustrating a procedure of the method of separating plasma or serum.

FIG. 19 is a cross-sectional view taken along a line 19-19 of FIG. 18.

FIG. 20 is a view illustrating the procedure of the method of separating plasma or serum.

FIG. 21 is a cross-sectional view taken along a line 21-21 of FIG. 20.

FIG. 22 is a view illustrating the procedure of the method of separating plasma or serum.

FIG. 23 is an assembly perspective view of the blood collection instrument and the separation and sealing instrument.

FIG. 24 is a view illustrating a procedure of the method of separating plasma or serum.

FIG. 25 is a view illustrating the procedure of the method of separating plasma or serum.

FIG. 26 is a view illustrating the procedure of the method of separating plasma or serum.

### DESCRIPTION OF THE PREFERRED EMBODIMENTS

Hereinafter, a package container, a sample examination kit, and a blood analysis method according to the present invention will be described with reference to the attached drawings. In the present invention, the numerical value range indicated by using “to” means a range including the numerical values before and after “to” as the lower limit value and the upper limit value, respectively.

<Blood Examination Kit>

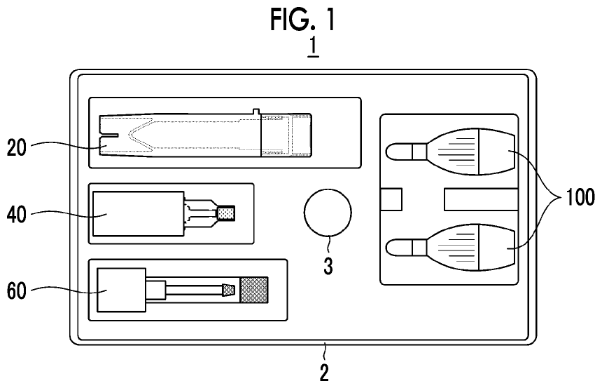

The blood examination kit of the embodiment will be described. FIG. 1 is a configuration view illustrating an example of a blood examination kit. As illustrated in FIG. 1, the blood examination kit 1 includes a blood collection instrument 20, an absorption instrument 40, a separation and sealing instrument 60, and a lancet 100. The blood collection instrument 20, the absorption instrument 40, the separation and sealing instrument 60, and the lancet 100 are accommodated in a case 2. A depression corresponding to the size of the blood collection instrument 20, the absorption instrument 40, the separation and sealing instrument 60, and the lancet 100 is formed in the case 2. The depression regulates the movement of the blood collection instrument 20, the absorption instrument 40, the separation and sealing instrument 60, and the lancet 100 during storage and transportation. In the present specification, the blood examination kit 1 includes a kit that is used for the examination and a kit that is used for collecting, separating, and storing the blood for use in the examination. The blood examination kit 1 does not need to have an examination function by itself.

A bottle stand 3 is formed substantially in the center of the case 2. The bottle stand 3 is a cylindrical depression into which the blood collection instrument 20 can be inserted. The bottle stand 3 can stand the blood collection instrument 20. The blood examination kit 1 can include an adhesive plaster, a disinfectant cloth, or an instruction manual, which is not illustrated in the drawing.

Hereinafter, each configuration of the blood examination kit 1 will be described.

<Blood Collection Instrument>

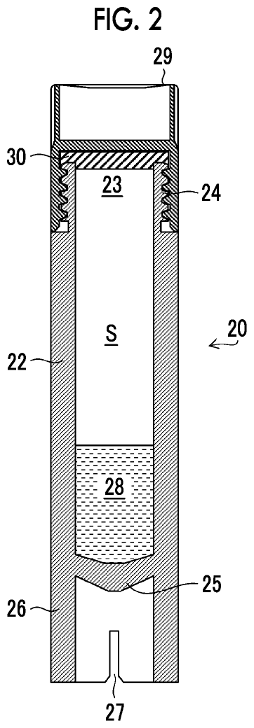

FIG. 2 is a cross-sectional view of the blood collection instrument 20 cut along the longitudinal direction. As illustrated in FIG. 2, the blood collection instrument 20 has a cylindrical first container 22 made of a transparent material. An opening 23 is formed on one end side of the first container 22. The first container 22 has a screw part 24 formed on the outer circumference surface of the end part of the opening 23. The first container 22 has a conical bottom part 25 on the other end side. An inner space S is formed in the first container 22. The bottom part 25 protrudes toward the other end side. The first container 22 has a cylindrical leg part 26 formed around the bottom part 25.

The leg part 26 has the same outer diameter as the sample cup (not illustrated in the drawing) that is used during the blood analysis examination. In the leg part 26, a plurality of slit grooves 27 that are arranged to face each other are preferably provided. The slit groove 27 is opened on the other end side and extends in the longitudinal direction.

A diluent 28 is accommodated in the bottom part 25 of the first container 22. The required amount of the diluent 28 is, for example, 500 mm³.

As illustrated in FIG. 2, before using the blood collection instrument 20, it is preferable that the opening 23 of the first container 22 is sealed by a cap 29 through a packing 30. In a case of collecting blood, the cap 29 is removed.

The diluent 28 is not particularly limited; however, for example, the diluent described in JP2017-01571A can be applied.

Blood can be collected in the blood collection instrument 20 by using an absorption instrument 40 described later.

<Absorption Instrument>

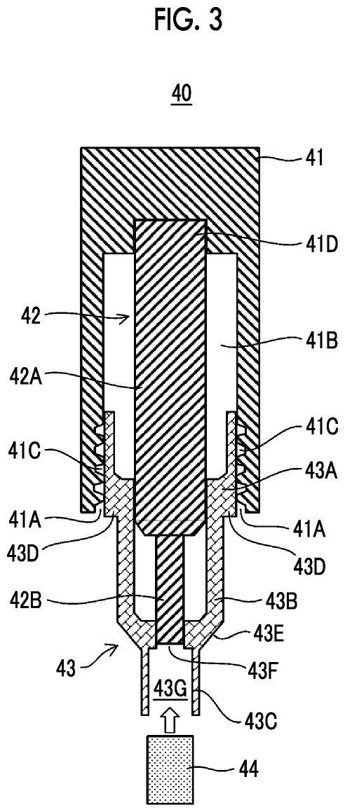

FIG. 3 is a cross-sectional view of the absorption instrument 40 cut along the longitudinal direction. As illustrated in FIG. 3, the absorption instrument 40 includes a lid portion member 41 which is a first lid portion member, arranged on one end side. The lid portion member 41 is configured to seal the opening 23 of the first container 22 of the blood collection instrument 20. In the lid portion member 41, an opening 41A is formed on the other end side, and the lid portion member 41 has a bottomed cylindrical shape in which an inner space 41B communicating with the opening 41A is formed. A screw part 41C that is screwable to the screw part 24 of the blood collection instrument 20 is provided on the inner circumference surface of the end part of the opening 41A of the lid portion member 41. A cylindrical depression 41D is provided on one end side of the inner space 41B of the lid portion member 41.

A protruding shaft 42 extending from one end side toward the other end side is fitted into the depression 41D and fixed. The protruding shaft 42 includes a large diameter part 42A that is arranged on one end side and a small diameter part 42B that is connected to the large diameter part 42A and extends to the other end side. The diameter of the small diameter part 42B is smaller than the diameter of the large diameter part 42A.

The absorption instrument 40 includes a holding member 43 that protrudes from the opening 41A and extends to the other end side. The holding member 43 includes, from one end side to the other end side, a large diameter part 43A, a medium diameter part 43B having a diameter smaller than that of the large diameter part 43A, and a small diameter part 43C having a diameter smaller than that of the medium diameter part 43B. The large diameter part 43A, the medium diameter part 43B, and the small diameter part 43C internally communicate from one end side to the other end side. A flange part 43D is formed in the holding member 43 due to the difference in diameter between the large diameter part 43A and the medium diameter part 43B.

The size of the outer diameter of the large diameter part 43A of the holding member 43 is substantially the same as the size of the inner diameter of the lid portion member 41. The large diameter part 43A is fitted into the inner space 41B. The large diameter part 43A is held by the frictional force with the inner surface of the lid portion member 41. The holding member 43 is slidably held in the inner space 41B of the lid portion member 41.

“Slidably held” means a state that (1) in a case where no external force is applied to any one of the holding member 43 and the lid portion member 41, the holding member 43 and the lid portion member 41 maintain their positional relationship by frictional force, and (2) in a case where an external force is applied to at least one of the holding member 43 or the lid portion member 41, the holding member 43 and the lid portion member 41 is relatively movable against the frictional force.

The inner diameter of the medium diameter part 43B of the holding member 43 is larger than the outer diameter of the large diameter part 42A of the protruding shaft 42. The medium diameter part 43B of the holding member 43 slidably accommodates the large diameter part 42A of the protruding shaft 42.

The medium diameter part 43B of the holding member 43 has a taper part 43E of which the diameter is reduced toward the small diameter part 43C. An opening 43F through which the small diameter part 42B of the protruding shaft 42 can pass is formed in the taper part 43E.

An absorption member 44 is accommodated in an inner space 43G of the small diameter part 43C. The size of the inner diameter of the inner space 43G is smaller than the size of the outer diameter of the absorption member 44, and thus the absorption member 44 is slidably held in the inner space 43G of the holding member 43 due to the frictional force on the inner surface of the small diameter part 43C.

“Slidably held” means a state that (1) in a case where no external force is applied to any one of the holding member 43 and the absorption member 44, the holding member 43 and the absorption member 44 maintain their positional relationship by frictional force, and (2) in a case where an external force is applied to at least one of the holding member 43 or the absorption member 44, the holding member 43 and the absorption member 44 is relatively movable against the frictional force.

The holding member 43 is preferably transparent. The transparency of the holding member 43 makes the state of the absorption member 44 visible to an examination subject.

The absorption member 44 is a fiber rod and is composed of, for example, a fiber. The fiber is preferably a synthetic fiber and more preferably a polyester fiber. In a case of a polyester fiber, the shape of the fiber can be easily changed. The absorption member 44 can be composed of a plurality of fibers having different fiber diameters. The absorption member 44 preferably has a void ratio in a range of 90% to 97%. In this case, the absorption of the blood sample becomes easier.

<Separation and Sealing Instrument>

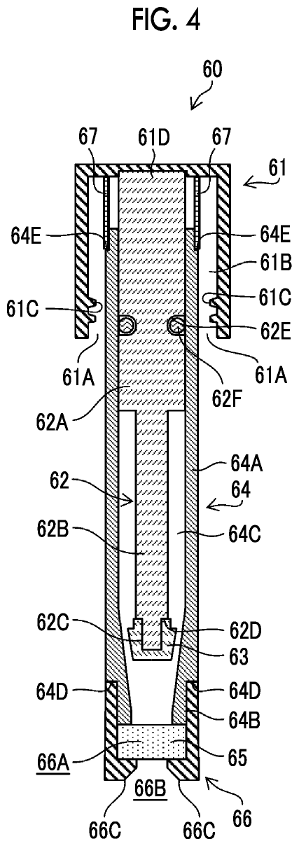

FIG. 4 is a cross-sectional view of the separation and sealing instrument 60 cut along the longitudinal direction. The separation and sealing instrument 60 includes a lid portion member 61 which is a second lid portion member, arranged on one end side. The lid portion member 61 is configured to seal the opening 23 of the first container 22 of the blood collection instrument 20. In the lid portion member 61, an opening 61A is formed on the other end side, and the lid portion member 61 has a bottomed cylindrical shape in which an inner space 61B communicating with the opening 61A is formed. A screw part 61C that is screwable to the screw part 24 of the blood collection instrument 20 is provided on the inner circumference surface of the end part of the opening 61A of the lid portion member 61. A cylindrical depression 61D is provided on one end side of the inner space 61B of the lid portion member 61.

A rod member 62 extending from one end side toward the other end side is fitted into the depression 61D and fixed. The rod member 62 includes, from one end side to the other end side, a large diameter part 62A, a medium diameter part 62B having a diameter smaller than that of the large diameter part 62A, and a small diameter part 62C having a diameter smaller than that of the medium diameter part 62B. A flange part 62D is formed in the rod member 62 due to the difference in diameter between the medium diameter part 62B and the small diameter part 62C.

As illustrated in FIG. 4, a groove 62E is formed on the outer circumference surface of the large diameter part 62A. A ring member 62F made of an elastic member is fitted into the groove 62E.

A valve member 63 is attached to cover the small diameter part 62C. The valve member 63 comes into contact with the flange part 62D, and the flange part 62D regulates the movement of the valve member 63 to one end side. The valve member 63 is made of, for example, silicone rubber. The valve member 63 has an inclined surface that expands from the other end side to the one end side.

The separation and sealing instrument 60 includes a second container 64 that can be inserted into the first container 22 of the blood collection instrument 20. The second container 64 is preferably made of a transparent member. The second container 64 has a substantially cylindrical shape and includes a large diameter part 64A and a small diameter part 64B having a diameter smaller than that of the large diameter part 64A. An inner space 64C that communicates one end side and the other end side is formed in the second container 64. The inner diameter of the second container 64 is reduced toward the opening of the small diameter part 64B. A flange part 64D is formed in the second container 64 due to the difference in diameter between the large diameter part 64A and the small diameter part 64B.

The rod member 62 is accommodated in the inner space 64C of the second container 64. The ring member 62F provided in the large diameter part 62A of the rod member 62 comes into contact with the inner surface of the second container 64. The rod member 62 is slidably held in the second container 64 by the frictional force with the inner surface of the second container 64.

A filtration member 65 is held on the other end side of the second container 64 so that the opening of the second container 64 is closed. As the filtration member 65, it is preferable to use a hydrophilic filter since it is necessary to filter a blood sample. Specifically, it is preferable to use a depth filter. The filtration member 65 can be composed of two or more depth filters. The filtration member 65 may be composed of two pieces of filters, a depth filter and a membrane filter. The filtration member 65 separates plasma or serum from a blood sample diluted with the diluent 28.

In a case where a blood sample is passed through the filtration member 65, the filtration member 65 separates plasma or serum from the blood sample. The filtration member 65 allows plasma or serum to pass through and blocks the passage of blood cells. The plasma or serum which has passed through the filtration member 65 to be separated is accommodated in the second container 64.

A sealing member 66 is mounted on the outer circumference of the small diameter part 64B. The sealing member 66 has a substantially cylindrical shape, and an inner space 66A that communicates one end side and the other end side is formed in the sealing member 66. A protruding portion 66C that defines an inflow port 66B is provided on one end side of the sealing member 66. The protruding portion 66C protrudes inward in the radial direction.

The filtration member 65 is sandwiched between one end of the second container 64 and the protruding portion 66C of the sealing member 66, and the filtration member 65 is held in the inner space 66A of the sealing member 66.

A spacer 67 is arranged between the lid portion member 61 and the second container 64. One end side of the spacer 67 is fixed to the lid portion member 61, and the other end side of the spacer 67 is in contact with the flange part 64E of the second container 64.

<Lancet>

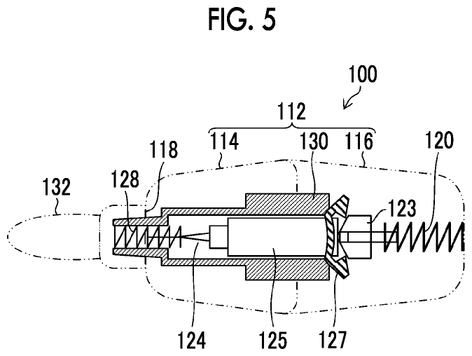

FIG. 5 is a cross-sectional view of the lancet. The lancet 100 is composed of a tip body part 114 and a base end body part 116 that form a lancet main body 112. An opening 118 is provided on the tip side of the tip body part 114.

The lancet 100 includes, from the side of the base end body part 116, a first elastic member 120, an engaging part 123, a fastener 127, a holding part 125, a puncture needle 124, a second elastic member 128, and a cap 132. A holder 130 accommodates the holding part 125, the puncture needle 124, and the second elastic member 128.

Before use, the first elastic member 120 is in a stretched state. The cap 132 is removed, and the lancet main body 112 presses the puncture needle 124 toward a finger or the like. At that time, the first elastic member 120 is fastened by the fastener 127. Next, the fastener 127 is released by the force of the first elastic member 120, and the puncture needle 124 is pushed out toward the finger or the like by the first elastic member 120. The puncture needle 124 pops out from the lancet main body 112 and punctures a finger or the like. Immediately after the puncture needle 124 punctures the finger or the like, the second elastic member 128 pushes back the puncture needle 124 into the lancet main body 112.

In a case where the fastener 127 is released, the first elastic member 120 cannot be fastened, and the lancet 100 is in a state where the puncture needle 124 does not pop out from the lancet main body 112.

<Method of Separating Plasma or Serum>

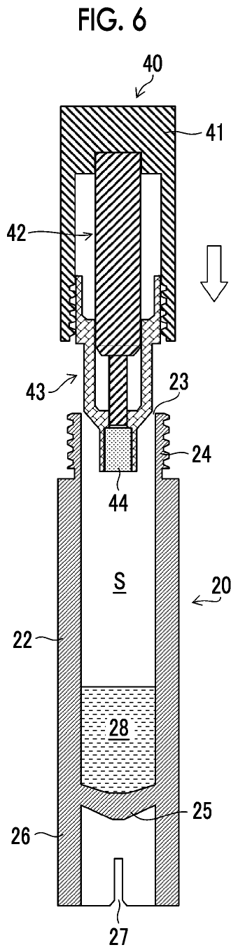

Next, a method of separating plasma or serum using the blood examination kit 1 will be described with reference to FIG. 6 to FIG. 13.

First, the cap 29 (not illustrated in the drawing) is removed from the blood collection instrument 20. The first container 22 is fitted into the bottle stand 3 of the case 2 (not illustrated in the drawing). The blood collection instrument 20 is prepared.

A finger or the like is punctured using the lancet 100, and a blood sample is squeezed out. The absorption member 44 of the absorption instrument 40 is brought into contact with the blood sample, and thus the blood sample is absorbed by the absorption member 44.

As illustrated in FIG. 6, the absorption member 44 of the absorption instrument 40 is directed toward the opening 23 of the blood collection instrument 20. The absorption instrument 40 is moved toward the blood collection instrument 20 in the direction of the arrow.

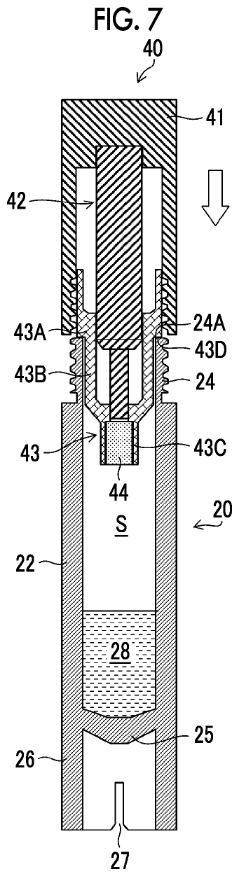

As illustrated in FIG. 7, the absorption instrument 40 is mounted on the blood collection instrument 20. The small diameter part 43C and the medium diameter part 43B that hold the absorption member 44 of the holding member 43 are accommodated in the inner space S of the first container 22.

On the other hand, since the flange part 43D of the holding member 43 comes into contact with one end 24A of the screw part 24 of the first container 22, the large diameter part 43A of the holding member 43 is not accommodated in the inner space S of the first container 22. The one end 24A of the first container 22 regulates the movement of the holding member 43 to the other end side.

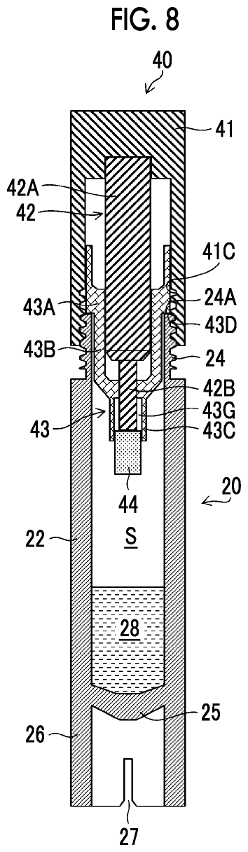

As illustrated in FIG. 8, the absorption instrument 40 is further moved toward the first container 22 of the blood collection instrument 20. The screw part 24 of the blood collection instrument 20 and the screw part 41C of the lid portion member 41 of the absorption instrument 40 are screwed with each other. The movement of the holding member 43 is regulated, whereas the movement of the lid portion member 41 is not regulated. In a case where the lid portion member 41 is screwed into the first container 22 by the screw part 41C and the screw part 24, the lid portion member 41 moves toward the side of the bottom part 25 of the blood collection instrument 20 against the frictional force with the holding member 43. Since the protruding shaft 42 is fitted and fixed to the lid portion member 41, the protruding shaft 42 moves toward the side of the bottom part 25 of the blood collection instrument 20 as the lid portion member 41 moves.

As illustrated in FIG. 8, the small diameter part 42B of the protruding shaft 42 pushes out the absorption member 44 from the inner space 43G of the holding member 43.

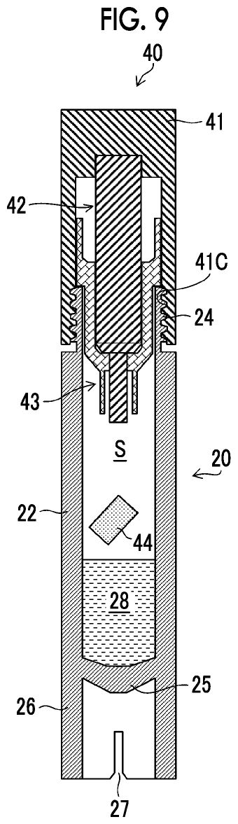

As illustrated in FIG. 9, the lid portion member 41 of the absorption instrument 40 seals the opening 23 of the first container 22. In the embodiment, the protruding shaft 42 pushes out the absorption member 44 from the holding member 43 toward the diluent 28.

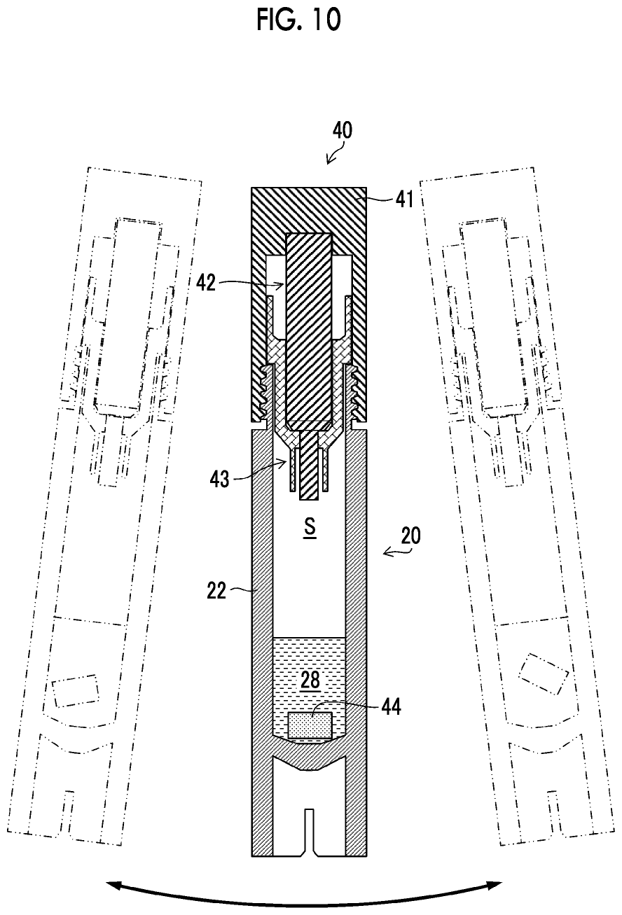

Next, as illustrated in FIG. 10, the absorption member 44 is immersed in the diluent 28. With the opening 23 (not illustrated in the drawing) of the first container 22 being sealed by the lid portion member 41 of the absorption instrument 40, an examination subject grasps the upper part of the first container 22 of the blood collection instrument 20 and then shakes the absorption instrument 40 and the blood collection instrument 20 several tens of times like a movement of a pendulum, whereby the blood sample is eluted from the absorption member 44 into the diluent 28.

In the embodiment, the absorption instrument 40 serves to provide (1) a function of absorbing the blood sample and (2) a function of a sealing lid that prevents the diluent 28 from leaking out in a case where the blood sample is eluted from the absorption member 44 into the diluent 28. With the absorption instrument 40, the work carried out by an examination subject can be simplified.

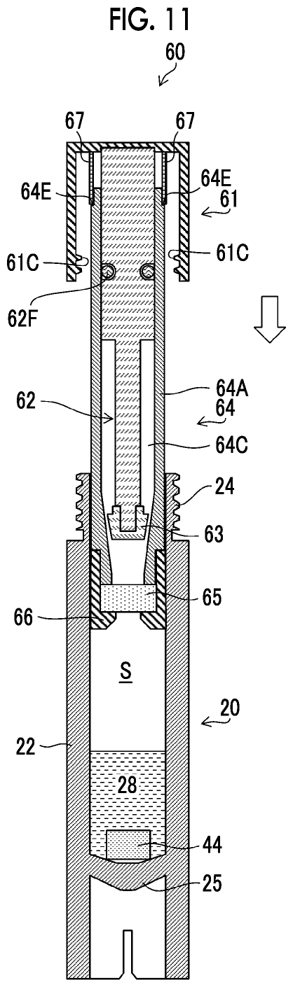

Next, the absorption instrument 40 is removed from the blood collection instrument 20. As illustrated in FIG. 11, the separation and sealing instrument 60 is moved toward the blood collection instrument 20. The filtration member 65 of the separation and sealing instrument 60 is directed toward the side of the blood collection instrument 20, and the second container 64 of the separation and sealing instrument 60 is inserted into the first container 22 of the blood collection instrument 20.

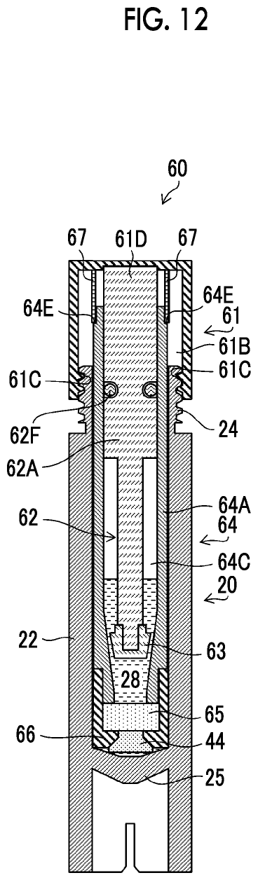

Next, as illustrated in FIG. 12, the separation and sealing instrument 60 is moved toward the blood collection instrument 20 until the other end side of the separation and sealing instrument 60 reaches the bottom part 25 of the blood collection instrument 20.

In a case where the second container 64 is inserted into the first container 22, the diluent 28 of the first container 22 passes through the filtration member 65 and move to the inner space 64C through the flow channel between the first container 22 and the second container 64. The second container 64 accommodates the plasma or serum separated by the filtration member 65. The filtration member 65 blocks the passage of the blood cells in the diluent 28.

The sealing member 66 attached to the second container 64 comes into contact with the inner surface of the first container 22 while the second container 64 inserts the first container 22. The sealing member 66 prevents the diluent 28 from leaking out between the inner surface of the first container 22 and the outer surface of the second container 64.

The screw part 24 of the blood collection instrument 20 and the screw part 61C of the lid portion member 61 of the separation and sealing instrument 60 are screwed with each other.

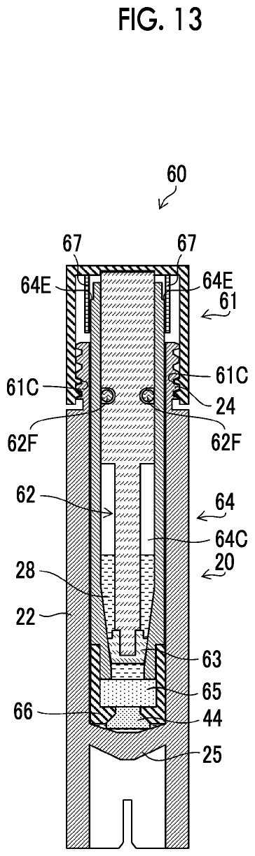

Finally, as illustrated in FIG. 13, in a case where the lid portion member 61 is screwed into the first container 22 by the screw part 61C of the lid portion member 61 and the screw part 24 of the blood collection instrument 20, the lid portion member 61 moves to the other end side. On the other hand, since the second container 64 has reached the bottom part 25 of the first container 22, the movement of the second container 64 is regulated. As a result, the spacer 67 fixed to the lid portion member 61 and in contact with the flange part 64E is destroyed. The contact between the flange part 64E and the spacer 67 is released.

After the second container 64 reaches the first container 22, the rod member 62 fixed to the lid portion member 61 move toward the other end side against the frictional force between the ring member 62F and the inner surface of the second container 64 as the lid portion member 61 moves. In a case where the lid portion member 61 seals the opening 23 (not illustrated in the drawing) of the first container 22, the valve member 63 closes the flow channel between the first container 22 and the second container 64. The valve member 63 prevents the filtered diluent 28 (the plasma or serum diluent) from flowing back to move from the side of the second container 64 to the side of the first container 22. The blood collection instrument 20 is sealed by the separation and sealing instrument 60 and mailed to the examination institution in the sealed state.

In the embodiment, the separation and sealing instrument 60 serves to provide (1) a function of separating plasma or serum from the blood sample and (2) a function of a sealing lid that prevents the diluent filtered from the blood collection instrument 20 from leaking out. With the separation and sealing instrument 60, the work carried out by an examination subject can be simplified.

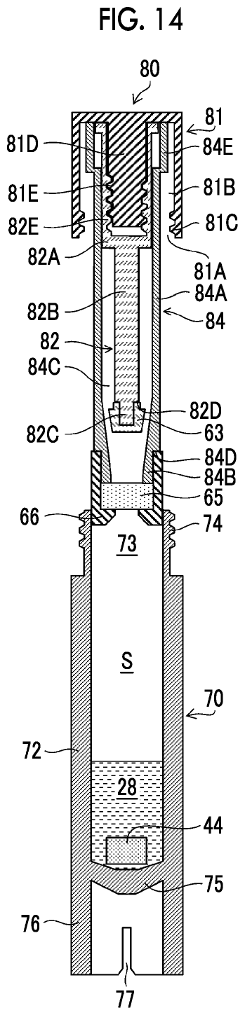

Next, the structure of another aspect of the separation and sealing instrument and another aspect of the method of separating plasma or serum will be described with reference to FIG. 14 to FIG. 16.

The blood collection instrument 70 has a cylindrical first container 72 made of a transparent material. An opening 73 is formed on one end side of the first container 72. The first container 72 has a screw part 74 formed on the outer circumference surface of the end part of the opening 73. The first container 72 has a conical bottom part 75 on the other end side. An inner space S is formed in the first container 72. The bottom part 75 protrudes toward the other end side. The first container 72 has a cylindrical leg part 76 formed around the bottom part 75. In the leg part 76, a plurality of slit grooves 77 that are arranged to face each other are preferably provided. The diluent 28 is accommodated in the first container 72. The blood collection instrument 70 has basically the same configuration as the blood collection instrument 20. On the other hand, the screw structure is different between the screw part 74 and the screw part 24, and in the screw part 74, a screw is not formed on the other end side. The absorption member 44 is immersed in the diluent 28.

The separation and sealing instrument 80 includes a lid portion member 81 which is a second lid portion member, arranged on one end side. In the lid portion member 81, an opening 81A is formed on the other end side, and the lid portion member 81 has a bottomed cylindrical shape in which an inner space 81B communicating with the opening 81A is formed. A screw part 81C that is screwable to the screw part 74 of the blood collection instrument 70 is provided on the inner circumference surface of the end part of the opening 81A of the lid portion member 81. A protruding portion 81D extending to the other end side is provided in the inner space 81B of the lid portion member 81. A screw part 81E is provided in the outer circumference of the protruding portion 81D.

A rod member 82 extending from one end side toward the other end side is provided in the separation and sealing instrument 80. The rod member 62 includes, from one end side to the other end side, a large diameter part 82A, a medium diameter part 82B having a diameter smaller than that of the large diameter part 82A, and a small diameter part 82C having a diameter smaller than that of the medium diameter part 82B. A flange part 82D is formed in the rod member 62 due to the difference in diameter between the medium diameter part 62B and the small diameter part 62C. The valve member 63 is attached to cover the small diameter part 82C.

As illustrated in FIG. 14, an inner space having an opening on one end side is formed in the large diameter part 82A. A screw part 82E is formed on the inner surface of the large diameter part 82A. In a case where the screw part 82E and the screw part 81E are screwed with each other, the rod member 82 and the lid portion member 81 are screwably fixed. The screw part 82E and the screw part 81E have a reverse screw relationship with respect to the screw part 74 and the screw part 81C.

The separation and sealing instrument 80 includes a second container 84 that can be inserted into the first container 72 of the blood collection instrument 70. The second container 84 is preferably made of a transparent member. The second container 84 has a substantially cylindrical shape and includes a large diameter part 84A and a small diameter part 84B having a diameter smaller than that of the large diameter part 84A. An inner space 84C that communicates one end side and the other end side is formed in the second container 84. The inner diameter of the second container 64 is reduced toward the opening of the small diameter part 64B. A flange part 84D is formed in the second container 84 due to the difference in diameter between the large diameter part 84A and the small diameter part 84B. The second container 64 has an expanded diameter part 84E on one end side, and the expanded diameter part 84E is fixed to the lid portion member 81. The rod member 62 is accommodated in the inner space 84C of the second container 84.

A filtration member 65 is held on the other end side of the second container 64 so that the opening of the second container 64 is closed. A sealing member 66 is mounted on the outer circumference of the small diameter part 64B. The filtration member 65 is held by one end of the second container 64 and the sealing member 66.

The separation and sealing instrument 80 is moved toward the blood collection instrument 70. The filtration member 65 of the separation and sealing instrument 80 is directed toward the side of the blood collection instrument 70, and the second container 84 of the separation and sealing instrument 80 is inserted into the first container 72 of the blood collection instrument 70.

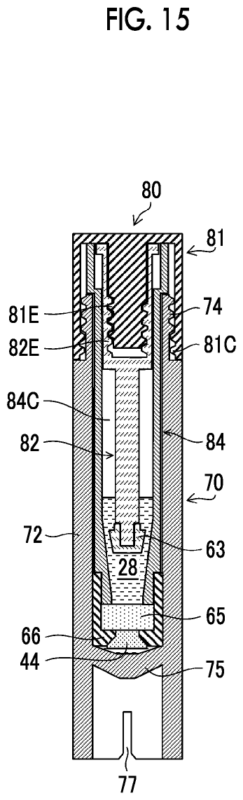

Next, as illustrated in FIG. 15, the lid portion member 81 is screwed into the first container 72 by the screw part 81C and the screw part 74. The separation and sealing instrument 80 is moved toward the blood collection instrument 70 until the other end side of the separation and sealing instrument 80 reaches the bottom part 75 of the blood collection instrument 70.

In a case where the second container 84 is inserted into the first container 72, the diluent 28 of the first container 72 passes through the filtration member 65. The diluent 28 moves to the inner space 84C through the flow channel between the first container 72 and the second container 84. The filtration member 65 separates plasma or serum from the blood sample. The filtration member 65 allows plasma or serum to pass through and blocks the passage of blood cells. The plasma or serum which has passed through the filtration member 65 to be separated is accommodated in the second container 84.

In a case where the other end side of the separation and sealing instrument 80 reaches the bottom part 75 of the blood collection instrument 70, the movement of the second container 84 (the movement to the other end side and the rotation around the longitudinal axis) is regulated. The screwing between the screw part 81C and the screw part 74 is released.

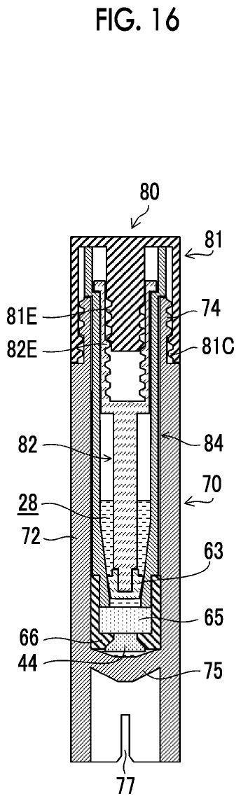

Finally, as illustrated in FIG. 16, in a case where the lid portion member 81 is rotated (idly rotated) after the second container 64 reaches the first container 22, the protruding portion 81D also rotates. On the other hand, the rotation of the rod member 82 screwed with each other with the protruding portion 81D is regulated by the frictional force with the inner surface of the second container 84 of which the movement is regulated.

In a case where the lid portion member 81 continues to rotate, since the screw part 82E of the rod member 82 and the screw part 81E of the lid portion member 81 have a reverse screw relationship, the rod member 82 moves in the direction in which the rod member 82 is spaced apart from the lid portion member 81, that is, toward the other end side.

The rod member 82 moves toward the other end side, and the valve member 63 closes the flow channel between the first container 72 and the second container 84. The movement of the rod member 82 is regulated, and the lid portion member 81 seals the opening 73 (not illustrated in the drawing) of the first container 72. The blood collection instrument 70 is sealed by the separation and sealing instrument 80 and mailed to the examination institution in the sealed state.

In the embodiment, the separation and sealing instrument 80 serves to provide (1) a function of separating plasma or serum from the blood sample and (2) a function of a sealing lid that prevents the diluent filtered from the blood collection instrument 70 from leaking out. With the separation and sealing instrument 80, the work carried out by an examination subject can be simplified.

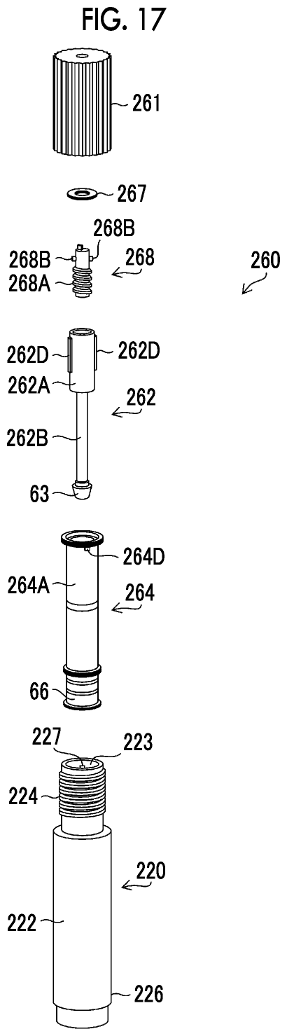

Next, the structure of another aspect of the separation and sealing instrument and another aspect of the method of separating plasma or serum will be described with reference to FIG. 17 to FIG. 22. In the following description of the separation and sealing instrument and the separation method, the description of the diluent 28 and the absorption member 44 will be omitted.

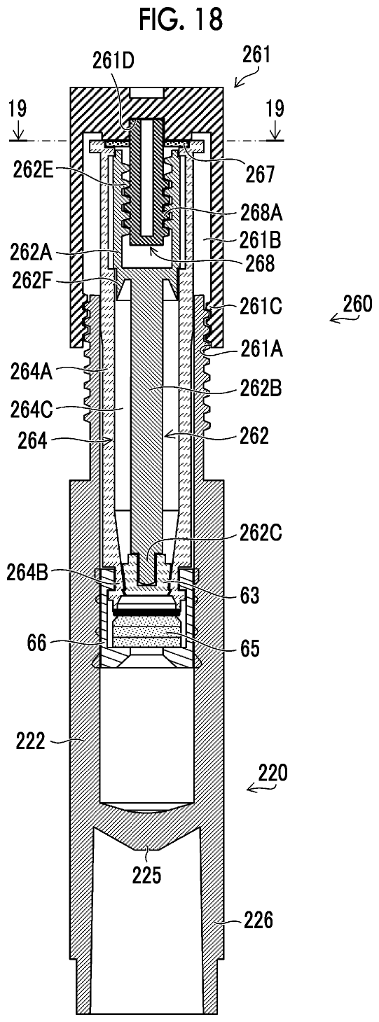

As illustrated in FIG. 17, the blood collection instrument 220 has a cylindrical first container 222 made of a transparent material. An opening 223 is formed on one end side of the first container 222. The first container 222 has a screw part 224 formed on the outer circumference surface of the end part of the opening 223. The screw part 224 is composed of a screw right-handed from one end side toward the other end side. On the outer circumference surface of the end part of the opening 223, the screw part 224 is not formed on the other end side. A conical bottom part 225 (see FIG. 18) is provided in the first container 222 on the other end side. The first container 222 has a cylindrical leg part 226 formed around the bottom part 225. A protruding portion 227 that protrudes inward is provided in the opening 223.

The separation and sealing instrument 260 includes a lid portion member 261 which is a second lid portion member, arranged on one end side. A plurality of grooves are formed on the outer circumference surface of the lid portion member 261 from one end side toward the other end side. The lid portion member 261 has a bottomed cylindrical shape in which an opening is formed on the other end side. The plurality of grooves of the lid portion member 261 facilitate attachment and detachment by an electric tool (not illustrated in the drawing).

The separation and sealing instrument 260 has a shaft member 268. In the inner space of the lid portion member 261, the shaft member 268 is fixed to the lid portion member 261. A screw part 268A is provided in the shaft member 268 in the outer circumference on the other end side. Unlike the screw part 224, the screw part 268A is composed of a reverse screw. The shaft member 268 has a pair of protruding portions 268B protruding outward, on the outer circumference on one end side.

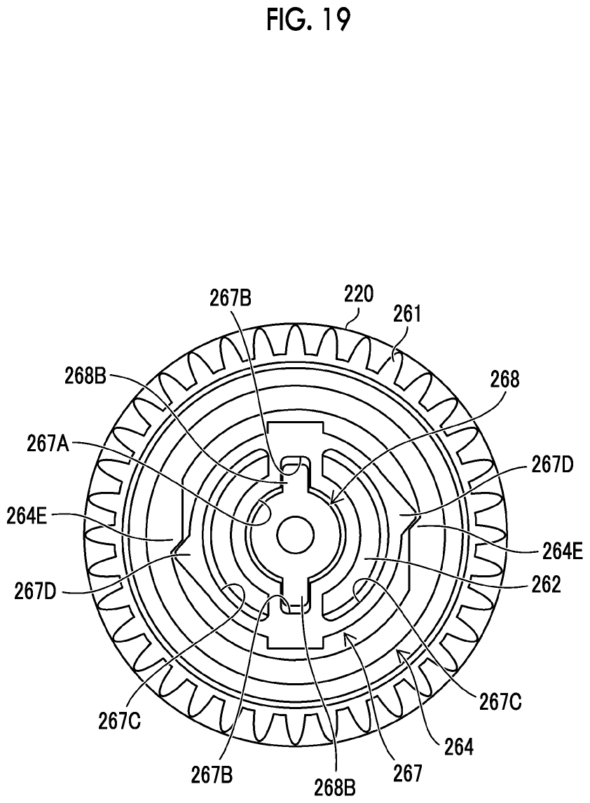

The separation and sealing instrument 260 has a power transmission member 267. The power transmission member 267 is engaged with the shaft member 268. The power transmission member 267 has a disk shape and is deformable inward in the radial direction. A groove 267B (see FIG. 19) formed in the power transmission member 267 engages with the protruding portion 268B of the shaft member 268. The power transmission member 267 is composed of at least one material selected from the group of polystyrene, polypropylene, and an acrylonitrile-butadiene-styrene copolymer.

The separation and sealing instrument 260 has a rod member 262. The rod member 262 includes, from one end side to the other end side, a large diameter part 262A, a medium diameter part 262B having a diameter smaller than that of the large diameter part 262A, and a small diameter part 262C (see FIG. 18) having a diameter smaller than that of the medium diameter part 262B. The valve member 63 is attached to cover the small diameter part 262C. The rod member 262 and the shaft member 268 are screwed with each other, and the rod member 262 and the shaft member 268 are screwably fixed. Two protruding portions 262D extending from one end side toward the other end side are formed on the outer circumference surface of the large diameter part 262A.

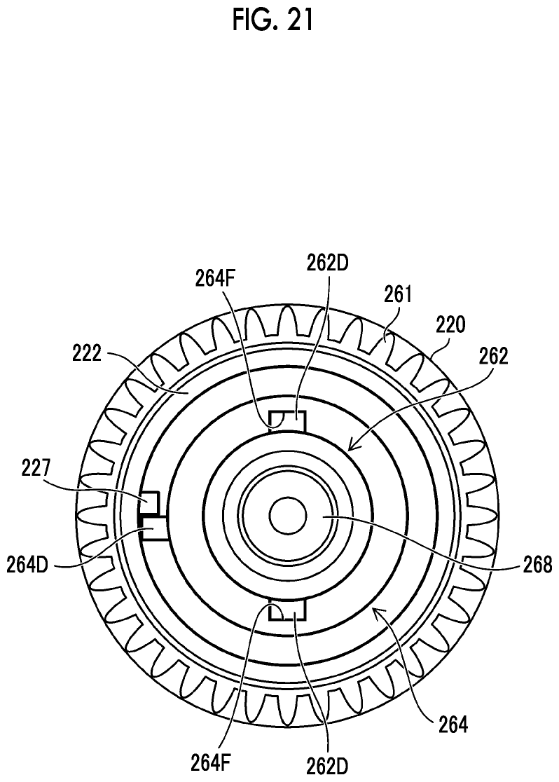

The separation and sealing instrument 260 includes a second container 264 that can be inserted into the first container 222 of the blood collection instrument 220. The second container 264 is preferably made of a transparent member. The second container 264 has a substantially cylindrical shape in which an inner space that communicates one end side and the other end side is formed. The second container 264 includes a large diameter part 264A and a small diameter part 264B (see FIG. 18) having a diameter smaller than that of the large diameter part 264A. The sealing member 66 is mounted so that the small diameter part 264B of the second container 264 is covered. The rod member 262 is accommodated in the inner space of the second container 264. The second container 264 includes a protruding portion 264D that protrudes toward the opening at one end in the radial direction. The protruding portion 264D engages with the protruding portion 227 of the first container 222 in the rotational direction. The second container 264 has a groove 264F (see FIG. 21) that accommodates the protruding portion 262D of the rod member 262 on the inner circumference surface thereof.

As illustrated in FIG. 18, in the separation and sealing instrument 260, the lid portion member 261, the power transmission member 267, the shaft member 268, the rod member 262, and the second container 264 are integrally assembled. In the lid portion member 261, an opening 261A is formed on the other end side, and the lid portion member 261 has a bottomed cylindrical shape in which an inner space 261B communicating with the opening 261A is formed. A screw part 261C that is screwable to the screw part 224 of the blood collection instrument 220 is provided on the inner circumference surface of the end part of the opening 261A of the lid portion member 261. A cylindrical depression 261D is provided on one end side of the inner space 261B of the lid portion member 261. The shaft member 268 is fitted into the depression 261D and fixed.

The rod member 262 includes the large diameter part 262A, the medium diameter part 262B, and the small diameter part 262C. An inner space having an opening on one end side is formed in the large diameter part 262A. A screw part 262E is formed on the inner surface of the large diameter part 262A. In a case where the screw part 262E of the rod member 262 and the screw part 268A of the shaft member 268 are screwed with each other, the rod member 262 and the shaft member 268 are screwably fixed. The screw part 262E and the screw part 268A have a reverse screw relationship with respect to the screw part 224 and the screw part 261C.

A taper shape part 262F tapered toward the other end side in the cross-sectional view is formed in the opening on the other end side of the large diameter part 262A.

The second container 264 includes the large diameter part 264A and the small diameter part 264B. An inner space 264C that communicates one end side and the other end side is formed in the second container 264. The filtration member 65 is held on the other end side of the second container 264 so that the opening of the second container 264 is closed. The sealing member 66 is mounted on the outer circumference of the small diameter part 264B. The filtration member 65 is held by one end of the second container 264 and the sealing member 66. The diameter of the large diameter part 264A is reduced toward the other end side at a position slightly on the other end side from the position of the taper shape part 262F.

The power transmission member 267 is fitted into the shaft member 268. The power transmission member 267 is sandwiched between the lid portion member 261 and the second container 264.

FIG. 19 is a cross-sectional view taken along the line 19-19 of FIG. 18. As illustrated in FIG. 19, the shaft member 268 has a pair of protruding portions 268B arranged to oppose each other.

The power transmission member 267 has a substantial disk shape in a plan view and has a through-hole 267A that accommodates the shaft member 268 and a groove 267B that engages with the protruding portion 268B. The plan view means a state where the power transmission member 267 is viewed in the axial direction of the through-hole 267A. The power transmission member 267 includes an arc-shaped through-holes 267C arranged with the through-hole 267A being interposed therebetween. The power transmission member 267 includes two protruding portions 267D arranged to oppose each other on the outer circumference surface. In a case where a force equal to or more than a predetermined force is applied toward the inside of the power transmission member 267 in the radial direction (crossing the through-hole 267C), the arc-shaped through-hole 267C is bent and deformed. The power transmission member 267 is elastically deformed as a whole.

The second container 264 includes a protruding portion 264E that protrudes inward in the radial direction, in the inner circumference of the opening at one end. As illustrated in FIG. 19, in the plan view, the two protruding portions 267D of the power transmission member 267 engages with the protruding portions 264E of the second container 264.

As illustrated in FIG. 18, the separation and sealing instrument 260 is moved toward the blood collection instrument 220. The filtration member 65 of the separation and sealing instrument 260 is directed toward the side of the blood collection instrument 220, and the second container 264 of the separation and sealing instrument 260 is inserted into the first container 222 of the blood collection instrument 220. The screw part 261C and the screw part 224 are screwed with each other, and the lid portion member 261 is screwed into the first container 222.

Next, the transmission of force will be described with reference to FIG. 19. The lid portion member 261 is rotated clockwise. Since the lid portion member 261 and the shaft member 268 are fixed, the shaft member 268 rotates clockwise together with the lid portion member 261. Since the protruding portion 268B of the shaft member 268 engages with the groove 267B of the power transmission member 267, the power transmission member 267 rotates clockwise together with the shaft member 268. Since the protruding portion 267D of the power transmission member 267 engages with the protruding portion 264E of the second container 264, the second container 264 rotates clockwise together with the power transmission member 267. Further, since the shaft member 268 and the rod member 262 are screwed with each other by the left-hand screw, the rod member 262 rotates clockwise together with the shaft member 268. The separation and sealing instrument 260 moves toward the other end side of the blood collection instrument 220 while rotating clockwise.

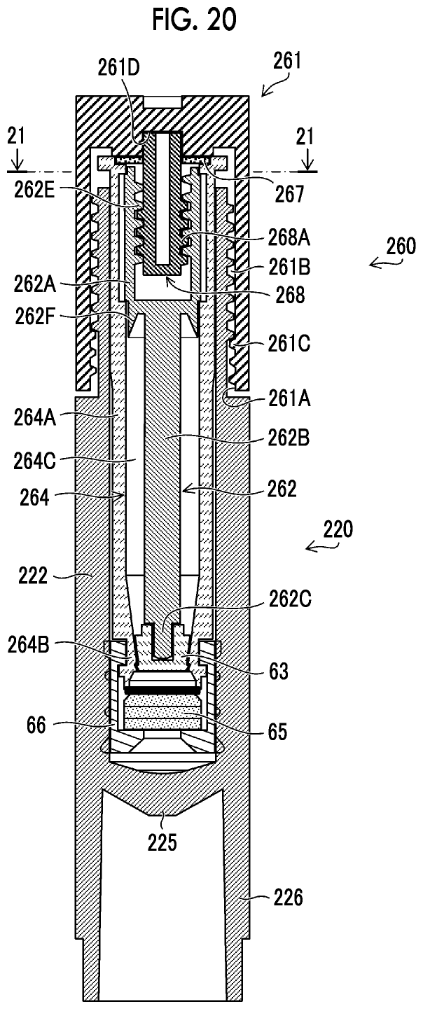

Next, as illustrated in FIG. 20, the separation and sealing instrument 260 moves toward the blood collection instrument 220 until the screw part 261C of the lid portion member 261 and the screw part 224 of the first container 222 are unscrewed (hereinafter, the movement end position). Although the diluent 28 and the absorption member 44 are not illustrated, the diluent 28 of the first container 222 passes through the filtration member 65, and the plasma or serum which has passed through the filtration member 65 to be separated is accommodated in the second container 264.

The sealing member 66 of the second container 264 comes into contact with the inner circumference surface of the first container 222 until the movement end position is reached. This contact provides resistance in a case where the power transmission member 267 rotates the second container 264 clockwise in FIG. 19. Since the second container 264 is rotated clockwise, torque is applied to the power transmission member 267. A component of a force of torque is applied toward the inside of the power transmission member 267 in the radial direction. In the embodiment, the power transmission member 267 does not deform due to having elastic force even in a case where a component of a force of torque is applied to the power transmission member 267 within the range of the torque applied to the power transmission member 267 until the movement end position is reached. The power transmission member 267 and the second container 264 rotate together until the movement end position is reached.

FIG. 21 is a cross-sectional view taken along the line 21-21 of FIG. 20. As illustrated in FIG. 21, at the movement end position, the protruding portion 227 of the first container 222 engages with the protruding portion 264D of the second container 264. In a case where the movement end position is reached, the rotation of the second container 264 is regulated. Since the protruding portion 262D of the rod member 262 engages with the groove 264F formed on the inner circumference surface of the second container 264, the rotation of the rod member 262 is regulated. In a case where the lid portion member 261 is further rotated clockwise (idly rotated) from the state of the movement end position, since the rotation of the second container 264 is regulated, a torque equal to or more than the torque applied until the movement end position is reached is applied to the power transmission member 267 in FIG. 19. In a case where a component of a force of torque is applied to the power transmission member 267, the arc-shaped through-hole 267C is bent and deformed, and thus the power transmission member 267 is elastically deformed as a whole. The protruding portion 267D of the power transmission member 267 gets over the protruding portion 264E of the second container 264 and rotates clockwise. The so-called power transmission member 267 starts idling rotation. As illustrated in FIG. 21, the rotation of the rod member 262 is regulated. In a case where the lid portion member 261, the shaft member 268, and the power transmission member 267 continue to rotate clockwise, since the shaft member 268 and the rod member 262 have a reverse screw relationship, the rod member 262 moves without rotation in the direction in which the rod member 262 is spaced apart from the shaft member 268, that is, toward the other end side. In a case where a torque of 0.1 Nm or more is applied to the power transmission member 267 in a state where the protruding portion 267D of the power transmission member 267 engages with the protruding portion 264E of the second container 264, it is preferable for the power transmission member 267 to deform elastically. For the torque value, a torque gauge (for example, product name “ATG/BTG (-S)”, manufactured by TOHNICHI Co., Ltd.) is attached to the center of the power transmission member 267, and the torque value can be measured from the maximum value (the rotation torque) of rotation of the power transmission member 267.

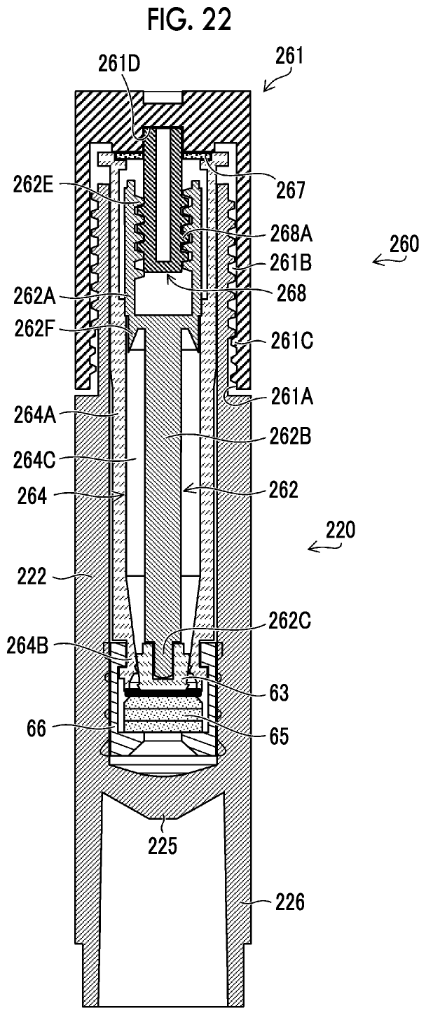

As illustrated in FIG. 22, the rod member 262 moves toward the other end side, and the valve member 63 closes the flow channel (hereinafter, the flow channel closing position) between the first container 222 and the second container 264. As a result, the movement of the rod member 262 to the other end side is regulated. In a case where the lid portion member 261 and the shaft member 268 are further rotated clockwise, the lid portion member 261 and the shaft member 268 move in the direction in which the lid portion member 261 and the shaft member 268 are spaced apart from the rod member 262, that is, toward one end side. The screw part 261C of the lid portion member 261 comes into contact with the screw part 224 of the first container 222, and the rotation of the lid portion member 261 is regulated. At the flow channel closing position, the taper shape part 262F of the rod member 262 is deformed inward along the reduced diameter shape of the large diameter part 264A of the second container 264. The taper shape part 262F prevents the plasma or serum accommodated in the second container 264 from leaking out from the second container 264. The blood collection instrument 220 is sealed by the separation and sealing instrument 260 and mailed to the examination institution in the sealed state.

In the embodiment, the separation and sealing instrument 260 serves to provide (1) a function of separating plasma or serum from the blood sample and (2) a function of a sealing lid that prevents the diluent filtered from the blood collection instrument 220 from leaking out. With the separation and sealing instrument 260, the work carried out by an examination subject can be simplified.

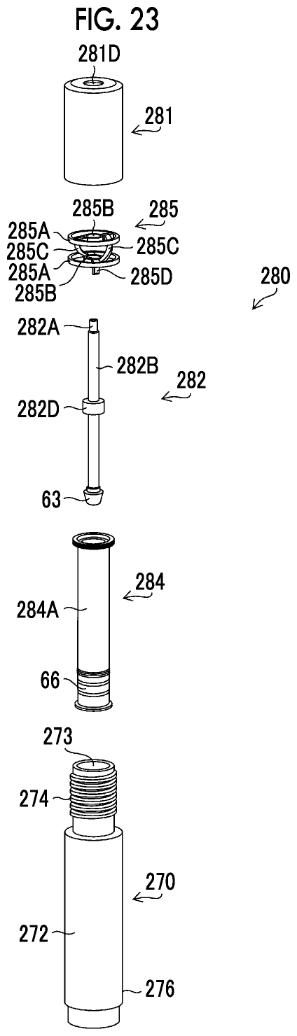

Next, the structure of another aspect of the separation and sealing instrument and another aspect of the method of separating plasma or serum will be described with reference to FIG. 23 to FIG. 26. In the following description of the separation and sealing instrument and the separation method, the description of the diluent 28 and the absorption member 44 will be omitted.

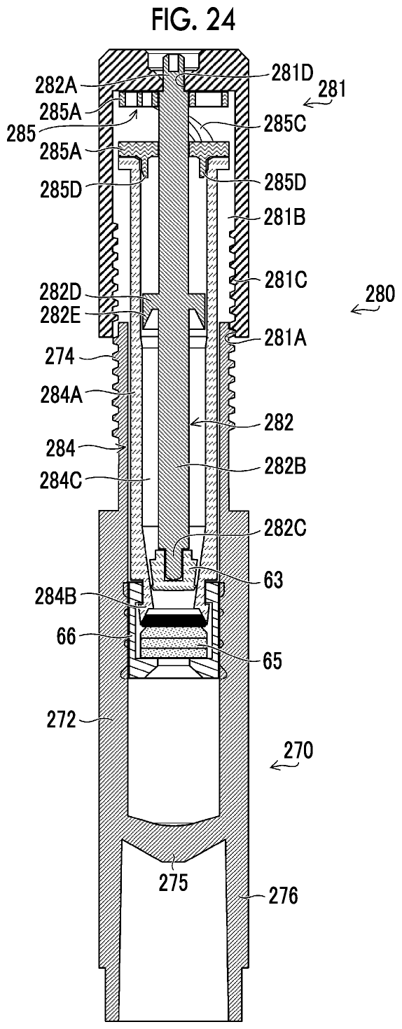

As illustrated in FIG. 23, the blood collection instrument 270 has a cylindrical first container 272 made of a transparent material. An opening 273 is formed on one end side of the first container 272. The first container 272 has a screw part 274 formed on the outer circumference surface of the end part of the opening 273. The screw part 274 is composed of a screw right-handed from one end side toward the other end side. On the outer circumference surface of the end part of the opening 273, the screw part 274 is not formed on the other end side. A conical bottom part 275 (see FIG. 24) is provided in the first container 272 on the other end side. The first container 272 has a cylindrical leg part 276 formed around the bottom part 275.

The separation and sealing instrument 280 includes a lid portion member 281 which is a second lid portion member, arranged on one end side. The lid portion member 281 has a bottomed cylindrical shape in which an opening is formed on the other end side. A through-hole 281D is formed in the lid portion member 281.

The separation and sealing instrument 280 has an elastic member 285. The elastic member 285 includes two disk-shaped members 285A in which a through-hole 285B is formed and a spring member 285C that mechanically connects the two disk-shaped members 285A. The elastic member 285 is a so-called compressive elastic member in which the height after compression becomes lower than the height of the natural length before compression in a case where a compressive force is applied in the axial direction of the through-hole 285B. The elastic member 285 is composed of at least one material selected from the group of polystyrene, polypropylene, and an acrylonitrile-butadiene-styrene copolymer. The spring constant of the elastic member 285 is preferably 4,900 N/m or more. The spring constant can be obtained from the ratio of the displacement amount of the compressive load to the displacement amount of the elastic member in a case where the compressive load is applied to the elastic member. The elastic member 285 has a protruding portion 285D that protrudes toward the other end side.

The separation and sealing instrument 280 has a rod member 282. The rod member 282 includes, from one end side to the other end side, a small diameter part 282A, a medium diameter part 282B having a diameter larger than that of the small diameter part 282A, and a small diameter part 282C (see FIG. 24) having a diameter smaller than that of the medium diameter part 282B. The medium diameter part 282B includes a large diameter part 282D at a substantially central part. The valve member 63 is attached to cover the small diameter part 282C. In the inner space of the lid portion member 281, the rod member 282 is fixed to the lid portion member 281.

The separation and sealing instrument 280 includes a second container 284 that can be inserted into the first container 272 of the blood collection instrument 270. The second container 284 is preferably made of a transparent member. The second container 284 has a substantially cylindrical shape in which an inner space that communicates one end side and the other end side is formed. The second container 284 includes a large diameter part 284A and a small diameter part 284B (see FIG. 24) having a diameter smaller than that of the large diameter part 284A. The sealing member 66 is mounted so that the small diameter part 284B of the second container 284 is covered. The rod member 282 is accommodated in the inner space of the second container 284.

As illustrated in FIG. 24, in the separation and sealing instrument 280, the lid portion member 281, the elastic member 285, the rod member 282, and the second container 284 are integrally assembled. In the lid portion member 281, an opening 281A is formed on the other end side, and the lid portion member 281 has a bottomed cylindrical shape in which an inner space 281B communicating with the opening 281A is formed. A screw part 281C that is screwable to the screw part 274 of the blood collection instrument 270 is provided on the inner circumference surface of the end part of the opening 281A of the lid portion member 281. A through-hole 281D is provided on one end side of the inner space 261B of the lid portion member 281. The small diameter part 282A of the rod member 282 is fitted into the through-hole 281D and fixed.

The rod member 282 includes the small diameter part 282A, the medium diameter part 282B, and the small diameter part 282C. A taper shape part 282E tapered toward the other end side in the cross-sectional view is formed in the large diameter part 282D provided in the medium diameter part 282B.

The second container 284 includes the large diameter part 284A and the small diameter part 284B. An inner space 284C that communicates one end side and the other end side is formed in the second container 284. The filtration member 65 is held on the other end side of the second container 284 so that the opening of the second container 284 is closed. The sealing member 66 is mounted on the outer circumference of the small diameter part 284B. The filtration member 65 is held by one end of the second container 284 and the sealing member 66. The diameter of the large diameter part 284A is reduced toward the other end side at a position slightly on the other end side from the position of the taper shape part 282E.

The elastic member 285 is fitted into the medium diameter part 282B of the rod member 282. The elastic member 285 and the second container 284 are connected by engaging the protruding portion 285D and the opening of the second container 284. The elastic member 285 is arranged between the lid portion member 281 and the second container 284.

As illustrated in FIG. 24, the separation and sealing instrument 280 is moved toward the blood collection instrument 270. The filtration member 65 of the separation and sealing instrument 280 is directed toward the side of the blood collection instrument 220, and the second container 284 of the separation and sealing instrument 280 is inserted into the first container 272 of the blood collection instrument 270. The screw part 281C and the screw part 274 are screwed with each other, and the lid portion member 281 is screwed into the first container 272.

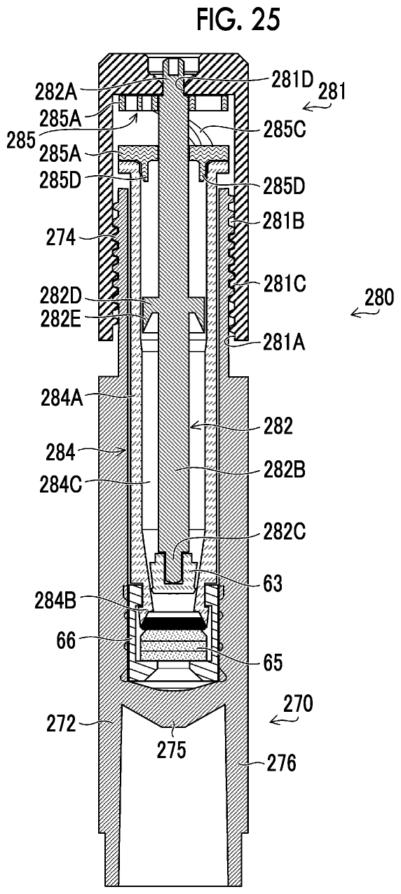

Next, as illustrated in FIG. 25, the separation and sealing instrument 280 is moved toward the blood collection instrument 270 until the other end side of the separation and sealing instrument 280 reaches the bottom part 275 (the movement end position) of the blood collection instrument 270. Although the diluent 28 and the absorption member 44 are not illustrated, the diluent 28 of the first container 272 passes through the filtration member 65, and the plasma or serum which has passed through the filtration member to be separated 65 is accommodated in the second container 284.

The sealing member 66 of the second container 284 comes into contact with the inner circumference surface of the first container 272 until the movement end position is reached. This contact provides resistance in a case where the second container 284 moves toward the first container 272. In a case where the lid portion member 281 is rotated clockwise to move the separation and sealing instrument 280, a compressive force is applied to the elastic member 285 arranged between the lid portion member 281 and the second container 284. In the embodiment, the elastic member 285 is set to a spring constant at which the elastic member 285 is not compressed by the compressive force applied until the movement end position is reached. As a result, the distance between the two disk-shaped members 285A of the elastic member 285 is constant until the movement end position is reached. “Not compressed” means that the ratio (H1/H) of the height (H1) of the elastic member 285 after compression to the height (H) of the natural length of the elastic member 285 is 0.95 or more.

In a case where the lid portion member 281 is further rotated clockwise from the state of the movement end position, since the screw part 281C of the lid portion member 281 and the screw part 274 of the first container 272 are screwed with each other, the lid portion member 281 moves toward the other end side. On the other hand, since the second container 284 has reached the bottom part 275, the movement of the second container 284 is regulated. Due to the movement of the lid portion member 281, a compressive force equal to or more than the compressive force applied until the movement end position is reached is applied to the elastic member 285. The elastic member 285 is compressed, and the rod member 282 moves toward the other end side.

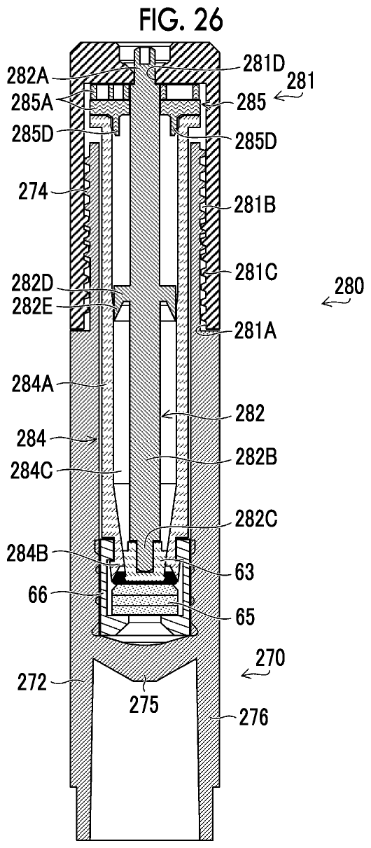

As illustrated in FIG. 26, two disk-shaped members 285A of the elastic member 285 are compressed until they come into contact with each other, the rod member 282 moves toward the other end side, and the valve member 63 closes the flow channel (hereinafter, the flow channel closing position) between the first container 272 and the second container 284. As a result, the movement of the rod member 282 to the other end side is regulated. At the flow channel closing position, the taper shape part 282E of the rod member 282 is deformed inward along the reduced diameter shape of the large diameter part 284A. The taper shape part 282E prevents the plasma or serum accommodated in the second container 284 from leaking out from the second container 284. The blood collection instrument 270 is sealed by the separation and sealing instrument 280 and mailed to the examination institution in the sealed state.

In the embodiment, the separation and sealing instrument 280 serves to provide (1) a function of separating plasma or serum from the blood sample and (2) a function of a sealing lid that prevents the diluent filtered from the blood collection instrument 270 from leaking out. With the separation and sealing instrument 280, the work carried out by an examination subject can be simplified.

### EXPLANATION OF REFERENCES

1: blood examination kit
2: case
3: bottle stand
20: blood collection instrument
22: first container
23: opening
24: screw part
24A: one end
25: bottom part
26: leg part
27: slit groove
28: diluent
29: cap
30: packing
40: absorption instrument
41: lid portion member
41A: opening
41B: inner space
41C: screw part
41D: depression
42: protruding shaft
42A: large diameter part
42B: small diameter part
43: holding member
43A: large diameter part
43B: medium diameter part
43C: small diameter part
43D: flange part
43E: taper part
43F: opening
43G: inner space
44: absorption member
60: separation and sealing instrument
61: lid portion member
61A: opening
61B: inner space
61C: screw part
62: rod member
62A: large diameter part
62B: medium diameter part
62C: small diameter part
62D: flange part
62E: groove
62F: ring member
63: valve member
64: second container
64A: large diameter part
64B: small diameter part
64C: inner space
64D: flange part
64E: flange part
65: filtration member
66: sealing member
66A: inner space
66B: inflow port
66C: protruding portion
67: spacer
70: blood collection instrument
72: first container
73: opening
74: screw part
75: bottom part
76: leg part
77: slit groove
80: separation and sealing instrument
81: lid portion member
81A: opening
81B: inner space
81C: screw part
81D: protruding portion
81E: screw part
82: rod member
82A: large diameter part
82B: medium diameter part
82C: small diameter part
82D: flange part
82E: screw part
84: second container
84A: large diameter part
84B: small diameter part
84C: inner space
84D: flange part
84E: expanded diameter part
100: lancet
112: lancet main body
114: tip body part
116: base end body part
118: opening
120: first elastic member
123: engaging part
124: puncture needle
125: holding part
127: fastener
128: second elastic member
130: holder
132: cap
220: blood collection instrument
222: first container
223: opening
224: screw part
225: bottom part
226: leg part
227: protruding portion
260: separation and sealing instrument
261: lid portion member
261A: opening
261B: inner space
261C: screw part
261D: depression
262: rod member
262A: large diameter part
262B: medium diameter part
262C: small diameter part
262D: protruding portion
262E: screw part
262F: taper shape part
264: second container
264A: large diameter part
264B: small diameter part
264C: inner space
264D: protruding portion
264E: protruding portion
264F: groove
267: power transmission member
267A: through-hole
267B: groove
267C: through-hole
267D: protruding portion
268: shaft member
268A: screw part
268B: protruding portion
270: blood collection instrument
272: first container
273: opening
274: screw part
275: bottom part
276: leg part
280: separation and sealing instrument
281: lid portion member
281A: opening
281B: inner space
281C: screw part
281D: through-hole
282: rod member
282A: small diameter part
282B: medium diameter part
282C: small diameter part
282D: large diameter part
282E: taper shape pan
284: second container
284A: large diameter part
284B: small diameter part
284C: inner space
285: elastic member
285A: disk-shaped member
285B: through-hole
285D: protruding portion
285C: spring member
S: inner space

What is claimed is:

1. A blood examination kit comprising:
a blood collection instrument including a diluent and a first container that has an opening formed on one end side and accommodates the diluent;
an absorption instrument including a first lid portion member that is arranged on one end side and can seal the opening of the first container, and an absorption member that is arranged on the other end side and absorbs a blood sample; and
a separation and sealing instrument including a filtration member that separates plasma or serum from the blood sample diluted with the diluent, a second container that holds the filtration member on the other end side, accommodates the separated plasma or serum, and can be inserted into the first container, a second lid portion member that is arranged on one end side of the second container and can seal the opening of the first container, and a valve member that closes a flow channel between the first container and the second container.

2. The blood examination kit according to claim 1,
the absorption instrument includes a holding member that holds the absorption member and a protruding shaft that is arranged on the one end side from the absorption member, and in a case where a first lid portion member of the absorption instrument seals the opening of the first container of the blood collection instrument, the protruding shaft pushes out the absorption member from the holding member into the diluent.

3. The blood examination kit according to claim 1,
wherein the absorption member of the absorption instrument is a fiber rod.

4. The blood examination kit according to claim 1,
wherein the separation and sealing instrument includes a rod member that holds the valve member on the other end side, and
after the second container of the separation and sealing instrument reaches the first container of the blood collection instrument, the second lid portion member of the separation and sealing instrument is rotated to move the rod member to a side of the filtration member, and the valve member that is held by the rod member closes the flow channel between the first container and the second container.

5. The blood examination kit according to claim 1,
wherein the filtration member of the separation and sealing instrument is hydrophilic.

6. The blood examination kit according to claim 1,
wherein the filtration member of the separation and sealing instrument includes a depth filter and a membrane filter.

7. The blood examination kit according to claim 1, further comprising a lancet having a puncture needle.

8. The blood examination kit according to claim 1,
wherein a power transmission member that transmits a force from the second lid portion member to the second container is provided, and
a protruding portion that is provided on the second container engages with a protruding portion that is provided on the power transmission member.

9. The blood examination kit according to claim 8,
wherein the power transmission member elastically deforms in a case where a torque of 0.1 Nm or more is applied.

10. The blood examination kit according to claim 1,
wherein a compressive elastic member is provided between the second container and the second lid portion member.

11. The blood examination kit according to claim 10,
wherein the compressive elastic member has a spring constant of 4,900 N/m or more.

12. A method of separating plasma or serum, comprising:
a step of preparing a blood collection instrument including a diluent and a first container that has an opening formed on one end side and accommodates the diluent;
a step of preparing an absorption instrument including a first lid portion member that is arranged on one end side and can seal the opening of the first container, and an absorption member that is arranged on the other end side and absorbs a blood sample and absorbing a blood sample into the absorption member;
a step of mounting the absorption instrument on the blood collection instrument, sealing the opening of the first container of the blood collection instrument with the first lid portion member of the absorption instrument, and eluting the blood sample from the absorption member into the diluent;
a step of removing the absorption instrument from the blood collection instrument; and
a step of preparing a separation and sealing instrument including a filtration member, a second container that holds the filtration member on the other end side and can be inserted into the first container, a second lid portion member that is arranged on one end side of the second container and can seal the opening of the first container, and a valve member that closes a flow channel between the first container and the second container, inserting the second container of the separation and sealing instrument into the first container of the blood collection instrument to separate plasma or serum from the blood sample diluted with the diluent with the filtration member, accommodating the separated plasma or serum in the second container, and sealing the opening of the first container of the blood collection instrument with second lid portion member of the separation and sealing instrument to close the flow channel between the first container and the second container with the valve member.

13. The method of separating plasma or serum according to claim 12,
wherein the step of eluting the blood sample includes pushing out the absorption member from the absorption instrument into the diluent.
